(19) United States
(12) Patent Application Publication
(10) Pub. No.: US 2012/0010523 A1
(43) Pub. Date: Jan. 12, 2012

(54) APPARATUS FOR DIAGNOSING FOOT NEUROPATHY AND METHOD OF USING THE SAME

(75) Inventors: Lorraine Ba-Tin, Wiltshire (GB); Charles Byrne, (US); Stacey Michael Finn, (US)

(73) Assignee: Salisbury NHS Foundation Trust

(21) Appl. No.: 13/041,438

(22) Filed: Mar. 7, 2011

(30) Foreign Application Priority Data
Jul. 6, 2010 (GB) 1011394.2

(51) Int. Cl. A61B 5/01

(52) U.S. Cl. 600/549

(57) ABSTRACT

An apparatus of use in identifying neuropathy in the feet of a subject is disclosed, the apparatus comprising means for measuring the temperature of the foot of a subject; means for comparing a first measured temperature obtained from one of the subject with a second measured temperature obtained from the other foot of the subject; and means for providing an indication to a user when the difference between the first and second measured temperatures exceeds a predetermined amount. A method method determining the relative temperature of the feet of a subject is also disclosed, the method comprising measuring the temperature of a first foot of the subject; measuring the temperature of a second foot of the subject; comparing the first measured temperature with the second measured temperature to determine the difference therebetween; and providing the user with an indication when the difference between the first and second measured temperatures exceeds a preset value.

The present invention relates to an apparatus for use in the diagnosis of neuropathy in the foot of a subject, in particular in diagnosing Charcot Foot. The present invention also relates to a method of using the same.

Neuropathy is a condition arising from damage to the nerves of a subject, in particular damage to the peripheral nerves, such as those in the limbs, especially the extremities of the limbs. Sufferers of diabetes are particularly susceptible to neuropathy, affecting the hands and feet. A very high proportion of diabetics exhibiting neuropathy develop the condition of Charcot Foot. Charcot Foot, also known as Charcot's arthropathy or Charcot neuroarthropathy, results from neuropathy in the feet of the subject and manifests itself as a weakening of the bones in the feet. Severe cases of Charcot Foot result in the bones becoming sufficiently weakened to fracture. As a result of the reduced nerve function, the sensations in the feet of the subject are reduced, in particular leading to a significant reduction in the sense of stimuli and pain. This leads to the ligaments, cartilage and bones in the feet being subjected to repeated trauma.

The condition of Charcot Foot generally affects just one foot of a sufferer, with both feet being affected in only 20% of cases. An early indication of the onset of Charcot Foot is a warming or increase in temperature of the foot, often accompanied by swelling and redness. As subluxation and dislocation of the joints occurs, the foot will become progressively deformed, with the bone developing deformities, such as protrusions.

The diagnosis of Charcot Foot may be difficult, particularly in its early stages. The loss of sensation in the foot of the subject arising from the neuropathy may be determined and devices are known to assist a clinician.

Thus, GB 2,410,028 discloses a monofilament device that may be used to test for diabetic neuropathy in a subject. In particular, the end of the monofilament may be applied to foot of the subject, to determine if the subject can sense the filament.

Further, devices comprising a fibre or monofilament are known and are commercially available. For example, the Neuropean® is a pen-shaped device comprising a monofilament to be applied at a predetermined pressure to the skin of a subject by a clinician. The device may be used in assessing a reduction in sensation to sharpness in small and large nerve fibres. A similar device is available commercially from Bailey Instruments Limited.

An alternative approach to determining the level of sensation in the feet of a subject is provided by the Tip Therm® device, also available from Bailey Instruments Limited. This device tests the sensitivity of the subject to temperature and applies a variable temperature to the skin of the subject to test the sensitivity to changes in temperature.

The Diaped Twin-Tip® device combines both a monofilament and a thermal device, for testing the sensitivity of the subject to both temperature and touch.

US 2005/0097762 discloses a system and method for examining a diabetic foot, in which an image of the sole of the foot is analysed. The system and method are indicated as being of use in the early assessment of neuropathy.

There is a need for an improved means for identifying the early onset of neuropathy, in particular Charcot Foot in a subject, in particular in a diabetic subject.

It has now been found that a simple and effective device for use by a clinician may be provided, the device being based on the detection of a significant temperature difference between the two feet of a subject. It has been found that a device providing a simple indication of the difference in temperature between the feet of a subject is effective in aiding a clinician in the early diagnosis of neuropathy and such conditions as Charcot Foot.

Accordingly, in a first aspect, the present invention provides an apparatus comprising:

means for measuring the temperature of the foot of a subject;

means for comparing a first measured temperature obtained from one of the subject with a second measured temperature obtained from the other foot of the subject; and

means for providing an indication to a user when the difference between the first and second measured temperatures exceeds a predetermined amount.

The apparatus of the present invention operates to provide the user with an indication of the temperature difference between the two feet of a subject. In particular, the apparatus is operable to provide an indication of a subject having feet differing in temperature by more than a predetermined or preset value. As described in more detail below, a significant difference in temperature between the feet of the subject may indicate neuropathy and, more particularly, a subject suffering from the early stages of Charcot Foot.

References herein to the temperature of the foot of a subject are references to the temperature of the surface or skin of the foot of the subject, as measured by a device responsive to the surface or skin temperature. This temperature is to be distinguished from a subcutaneous temperature or other temperature within the body of the subject.

The apparatus comprises a means for measuring the temperature of the foot of the subject. Any suitable means for measuring the foot temperature may be used, for example an analogue temperature sensor. Suitable means are known in the art and are commercially available. Preferably, the means for measuring the temperature is one that is applied to the foot of the subject and responds to the temperature of the skin of the subject, upon being applied thereto and being in contact therewith. Examples of suitable temperature sensing means include thermistors and resistive thermal devices (RTDs).

The means for measuring the temperature of the foot may have a wide operating range, that is measure a broad range of temperatures. However, as described in more detail below, the range of temperatures to be measured at or on the foot of the subject is relatively narrow, that is a temperature range above and below normal body temperature. Accordingly, the means for measuring temperature may have an operating range of at least body temperature +/−10° C., that is between 27° C. and 47° C., more preferably between 30° C. and 45° C., still more preferably between 35° C. and 43° C.

The means for measuring the temperature of the foot of the subject preferably responds quickly to the temperature being sensed, thereby reducing the length of time that the apparatus must be held at or in contact with the foot of the subject. The means for measuring the temperature may take some time to stabilise and provide a stable reading of the temperature. The length of time taken will depend upon the particular arrangement and the materials of construction employed. The time taken to reach a stable reading of the temperature may be up to 1 to 2 minutes. Preferably, the means for measuring the temperature of the foot of the subject is arranged to reduce the time taken to reach a stable temperature reading. Preferably, the temperature sensing means should respond within less than 60 seconds, more preferably less than 30 seconds. If the arrangement and materials allow, its preferred that the means for measuring the temperature provides a stable output signal in less than 10 seconds, more preferably less than 5 seconds, still more preferably within from 1 to 2 seconds.

In embodiments in which the means for measuring the temperatures of the feet of the subject are to be in contact with the skin of the subject, such as a thermistor or resistive thermal device, the means are preferably arranged to be easily cleaned or to be provided with a disposable cover or the like, to prevent the transfer of infections between subjects.

The apparatus of the present invention is used to measure the temperature of each foot of a subject and to detect a difference in temperature between the two feet. In use, the clinician uses the apparatus to measure the temperature of one foot, for example by placing the temperature sensing means in contact with the skin of the foot. The clinician then repeats the temperature measuring step on the second foot of the subject. The apparatus operates to compare the first and second temperature measurements, and thus compare the temperature of the feet of the subject. The clinician is provided with an indication of the temperature difference, as described in more detail below.

Accordingly, the apparatus of the present invention further comprises means for receiving data and/or signals from the temperatures sensing means and for comparing a first measured temperature and a second measured temperature. Any suitable means may be used to carry out the comparison of the first and second temperatures. In particular, the apparatus may comprise a suitable processor for receiving signals from the temperature sensing means relating to the first and second temperatures and for determining the difference between the first and second temperatures. Suitable processors are known in the art and are commercially available. One such processor is the 20-pin microcontroller PIC18LF14K22.

The apparatus further comprises means for providing an indication of the difference between the first measured temperature and the second measured temperature. In particular, the indication means provides the user with an indication when the difference between the first measured temperature and the second measured temperature exceeds a predetermined or preset value. In this respect, as described above, one symptom of neuropathy in a foot of a subject, in particular the onset of Charcot Foot, is that the temperature of the surface of the foot is elevated. As also noted, it is most commonly the case that Charcot Foot develops in just one foot of a subject. Thus, a temperature difference will occur between one foot of the subject and the other. The apparatus of the present invention is used to identify such a difference in temperature, thereby providing an indication that the subject may be suffering from neuropathy, in particular Charcot Foot, in the foot with the higher temperature. Any difference in temperature between the two feet of a subject may indicate neuropathy and Charcot Foot. However, it is generally understood that a temperature difference of 3° C. or greater is a strong indicator of neuropathy and Charcot Foot. Accordingly, the apparatus of the present invention is arranged to provide an indication of the difference between the first measured temperature, that is of the first foot when in use, and the second measured temperature, that is of the second foot when in use when the difference exceeds a predetermined or preset value, in particular of at least 1° C., more preferably at least 2° C., sill more preferably at least 3′C.

In use, the apparatus may measure and determine the absolute temperature of each foot of the subject, to thereby calculate the temperature difference between the feet. At the very least, the apparatus is arranged to detect a temperature difference between the two feet of the subject. In particular, the apparatus is preferably arranged to sense and record two stable temperatures, one from the surface of each foot of the subject person, and to calculate the difference between the two temperatures. Depending upon the order in which the temperatures of the feet are measured, that is either the coolest first or the warmest first, the difference in the temperatures may be either positive or negative. Preferably, the device determines the absolute difference between the two temperatures. In this way, the user is not required to identify the warmer or cooler foot of the subject, but may select either foot to begin the temperature measurement.

Any suitable indication means may be employed provided they provide the user with an indication of the difference between the two measured temperatures. In one embodiment, the indication means provides a value for the difference between the first and second measured temperatures, for example in ° C. More preferably, the indication means is operable to provide the user with a simple indication of whether the predetermined or preset temperature difference has been exceeded or not. The indication means may, for example, comprise an audible indication and/or a visual indication. For example, the indication means may comprise a sound generator and be operable to provide a first sound or tone when the measured temperature difference is less than the preset value and a second sound or tone when the measured temperature difference exceeds the preset value. Similarly, the indication means may comprise one or more lights, to provide a first visual indication, such as a light of a first colour, in cases where the measured temperature difference is less than the preset value and a second visual indication, such as a light of a second colour, when the preset value for the difference is exceeded. Light emitting diodes (LEDs) are particularly suitable and preferred indication means for providing the appropriate indication of the temperature difference to a user.

The apparatus may be operated automatically, for example when the temperature sensing means is contacted with the foot of a subject. More preferably, the apparatus is provided with a switch, allowing the user to selectively operate the apparatus when the first and second temperatures are to be measured and compared. The apparatus may comprise two or more switches, to provide control over the different functions of the device, for example a first switch to switch the device on, a second switch to select the temperature measurement, and a third switch to select the processing and/or output of data to the user. More preferably, the apparatus is provided with a single switch, the operation of which turns the device on to provide electrical power to the various components and to begin the sequence of temperature sensing and processing.

The apparatus may employ any suitable power source to power the components. Most preferably and conveniently, the apparatus is provided with a power storage means, in particular a battery, to provide electrical power to the components.

The apparatus may be arranged in any suitable form. It is an advantage of the apparatus of the present invention that it may be constructed from relatively few components and arranged in a compact configuration, for example to fit in the pocket of a clinician. In one preferred embodiment, the apparatus is arranged to be pen-like, so as to be held and carried in similar manner to a conventional pen.

As noted above, it is known in the art to determine the sensitivity of the skin of a subject to touch using a suitable device, such as a filament or the like. In one embodiment, the apparatus of the present invention further comprises a portion for contacting the skin of the subject, to allow the sensitivity of the skin of the subject to touch to be examined. The portion preferably comprises a mono-filament.

In a further aspect, the present invention provides a method determining the relative temperature of the feet of a subject, the method comprising:

measuring the temperature of a first foot of the subject:

measuring the temperature of a second foot of the subject;

comparing the first measured temperature with the second measured temperature to determine the difference therebetween; and

providing the user with an indication when the difference between the first and second measured temperatures exceeds a preset value.

Details of the method are as described hereinbefore. As noted, the preset value used in the method is a value of the temperature difference that is indicative of Charcot Foot, preferably at least 1° C., more preferably at least 2° C., still more preferably at least 3′C.

In a further aspect, the present invention provides a method of diagnosing neuropathy in a foot of a subject, the method comprising:

measuring the temperature of the foot of the subject;

measuring the temperature of the second foot of the subject;

comparing the first measured temperature with the second measured temperature to determine the difference therebetween; and

providing the user with an indication when the difference between the first and second measured temperatures exceeds a preset value.

Details of the method are as described hereinbefore. As noted, the preset value used in the method is a value of the temperature difference that is indicative of Charcot Foot, preferably at least 1° C., more preferably at least 2″C, still more preferably at least 3′C.

The present invention also provides the use of an apparatus as hereinbefore described in determining the difference in temperature between the feet of a subject.

The present invention further provides the use of an apparatus as hereinbefore described in diagnosing Charcot Foot.

Embodiments of the present invention will now be described, by way of example only, having reference to the accompanying drawings, in which:

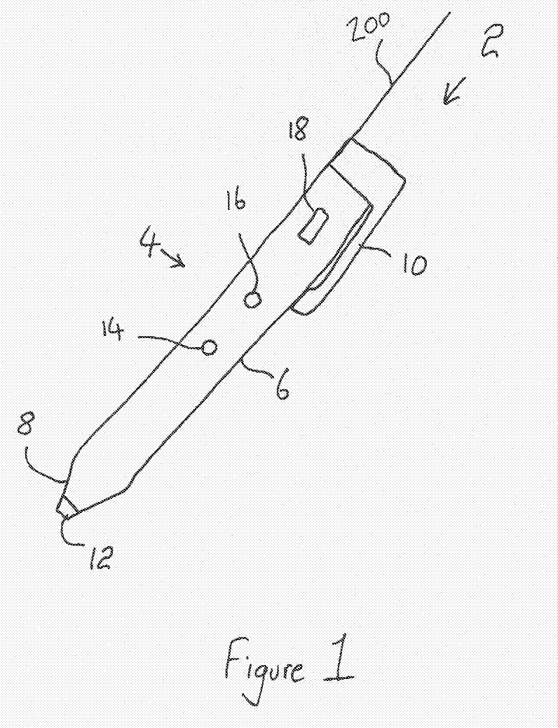

FIG. 1 is a general perspective view of an apparatus according to one embodiment of the present invention;

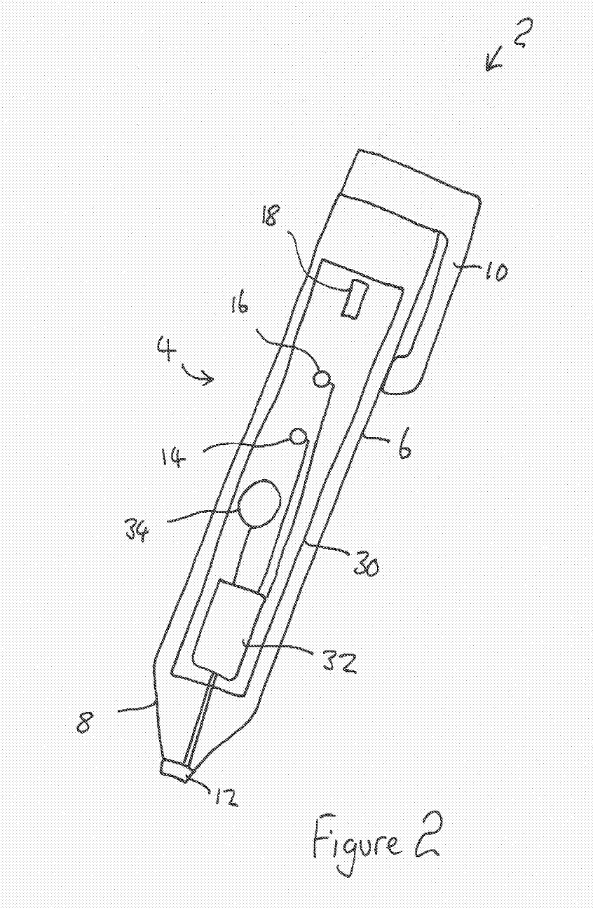

FIG. 2 is a cut-away view of the apparatus of FIG. 1 showing the major components of the apparatus; and

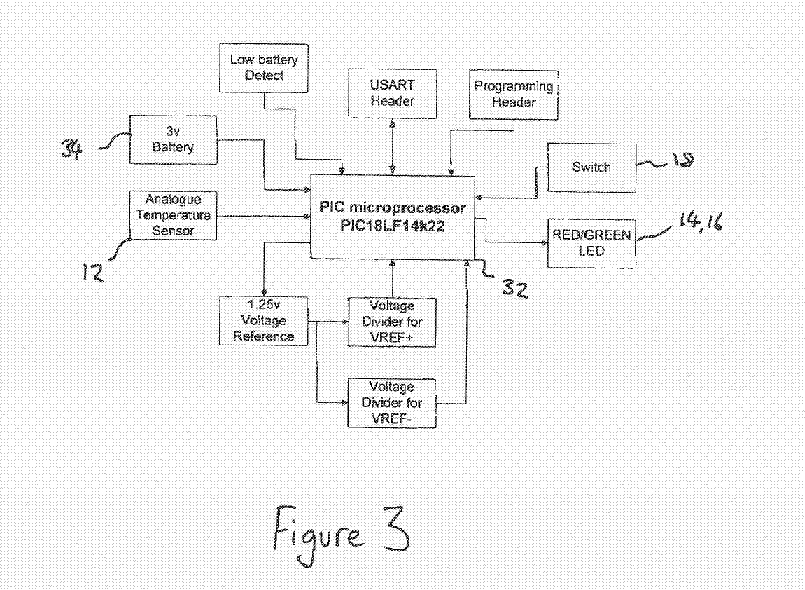

FIG. 3 is a schematic representation of the components of an apparatus of one embodiment of the present invention.

Turning to FIG. 1, there is shown a general perspective view of an apparatus according to one embodiment of the present invention and generally indicated as 2. The apparatus 2 comprises a generally pen-shaped housing 4, having a generally cylindrical body 6 and a tapered tip portion 8 at a first end of the body 6. A clip 10 is provided at the second end of the body 6, to allow the apparatus to be held in a pocket or the like, as is conventional with pens. The housing 4 may be formed from any suitable material, most preferably a suitable plastic.

A thermistor 12 is provided in the tapered tip portion 8 so as to extend from the tip, allowing the thermistor to be brought into contact with the skin of a foot of a subject. One preferred type of thermistor is a thermistor integrated circuit (IC). Such devices are known in the art and are available commercially, for example the range of linear active thermistor ICs from Microchip Technology Inc.

The body 6 is provided with a first, red LED 14 and a second green LED 16, providing an indication to the user of the difference between a first measured temperature and a second measured temperature relative to a preset difference value.

A switch 18 is provided in the body 6, to activate the device.

Referring to FIG. 2, there is shown a cutaway view of the apparatus 2 of FIG. 1, with a diagrammatical representation of components within the housing 4. As shown, the components are mounted in conventional manner on a printed circuit board (PCB) 30. The apparatus 2 comprises a processor 32, for receiving signals from the thermistor 12 when operated by the switch 18 and to make a comparison of the first and second measured temperatures. One suitable processor is the 20-pin microcontroller PIC18LF14K22. The processor 32 is powered by a battery 34 of conventional design and commercially available.

Referring to FIG. 3, there is a shown a schematic representation of the arrangement of the components of the apparatus with respect to the processor 32.

In use, the clinician places the tip 8 of the apparatus 2 against the skin of one foot of the subject and activates the processor using the switch 18. A first temperature measurement is made and recorded by the processor 32. The clinician then places the tip 8 against the skin of the second foot of the subject and activates the processor using the switch 18, to measure and record a second temperature. The processor 32 functions to compare the first and second measured temperatures and determine the difference therebetween. The processor 32 is provided with a predetermined or preset value for the temperature difference, preferably 3′C. If the difference between the first and second measured temperatures is less than 3° C., the processor operates to illuminate the green LED 16, indicating to the clinician that the difference in temperature between the feet of the subject is within an acceptable range. If the difference between the first and second measured temperatures is greater than 3° C., the processor 32 illuminates the red LED 14, indicating to the clinician that the subject has a foot with an elevated temperature, indicating a possible case of Charcot Foot.

The apparatus of this embodiment of the invention does not indicate to the user which foot of the subject is the warmer or the cooler. Rather, the apparatus determines the absolute temperature difference between the two feet of the subject and provides a display by way of the lights accordingly. Thus, in use, the user may begin the temperature sensing and processing task using either foot.

In an alternative embodiment, the apparatus is arranged to identify the higher and lower of the two temperature readings and to display to the user both an indication of the absolute difference in temperature and an indication whether the first or second sensed temperature is the higher or lower. By remembering or noting the first foot to be examined, the user may thus identify the warmer foot and cooler foot of the subject. This may assist in further diagnosis of the subject.

The apparatus shown in the figures may be provided with a mono-filament 200, as indicated in FIG. 1. The mono-filament 200 is mounted, for example pivotably mounted, to the body 6, so as to be moveable between a retracted position, in which the mono-filament is retained against or within the body 6, and an extended position, as shown in FIG. 1, in which the distal end of the mono-filament is exposed.

1. An apparatus comprising:
means for measuring the temperature of the foot of a subject;
means for comparing a first measured temperature obtained from one of the subject with a second measured temperature obtained from the other foot of the subject; and
means for providing an indication to a user when the difference between the first and second measured temperatures exceeds a predetermined amount.

2. The apparatus according to claim 1, wherein the means for measuring the temperature responds to the temperature when in contact with the skin of the foot of the subject.

3. The apparatus according to claim 2, wherein the means for measuring the temperature comprises a thermistor or a resistive thermal device.

4. The apparatus according to either of claim 2 or 3, wherein the means for measuring the temperature has a surface that is cleanable and/or wherein the said means is provided with a removable cover.

5. The apparatus according to any preceding claim, wherein the means for comparing the temperatures comprises a processor.

6. The apparatus according to any preceding claim, wherein the predetermined temperature difference is at least 3° C.

7. The apparatus according to any preceding claim, wherein the processor determines the absolute temperature difference between the first and second temperatures and operates the indication means to provide an indication if the absolute temperature difference exceeds the predetermined value.

8. The apparatus according to any preceding claim, wherein the processor determines which of the first and second temperatures is the higher, the indication means being operable to provide an indication of which temperature is the higher and/or which temperature is the lower.

9. The apparatus according to any preceding claim, wherein the indication means is operable to provide an indication to the user of the value of the difference in temperature between the first and second temperatures.

10. The apparatus according to any of claims 1 to 9, wherein the indication means is operable to provide an indication to the user only when the difference in temperature exceeds the predetermined amount.

11. The apparatus according to any preceding claim, wherein the indication means is operable to provide an audible indication.

12. The apparatus according to any preceding claim, wherein the indication means is operable to provide a visual indication.

13. The apparatus according to any preceding claim, wherein the indication means comprises at least one light emitting diode.

14. The apparatus according to any preceding claim, further comprising a self-contained electrical power source.

15. The apparatus according to any preceding claim, further comprising a monofilament.

16. The apparatus according to claim 15, wherein the monofilament is moveable between a retracted position and an extended position.

17. The apparatus according to any preceding claim, wherein the apparatus is pen-like.

18. A method determining the relative temperature of the feet of a subject, the method comprising:
measuring the temperature of a first foot of the subject;
measuring the temperature of a second foot of the subject;
comparing the first measured temperature with the second measured temperature to determine the difference therebetween; and
providing the user with an indication when the difference between the first and second measured temperatures exceeds a preset value.

19. The method according to claim 18, wherein the preset value is at least 3° C.

20. The method according to either of claim 18 or 19, wherein the user is provided with an indication of the value of the temperature difference.

21. The method according to either of claim 18 or 19, wherein the user is provided with an indication only when the temperature difference exceeds the preset value.

22. The method according to any of claims 18 to 21, wherein the user is provided with an audio and/or a visual indication.

23. A method of diagnosing neuropathy in a foot of a subject, the method comprising:
measuring the temperature of the foot of the subject;
measuring the temperature of the second foot of the subject;
comparing the first measured temperature with the second measured temperature to determine the difference therebetween; and
providing the user with an indication when the difference between the first and second measured temperatures exceeds a preset value.

24. The use of an apparatus according to any of claims 1 to 17 in diagnosing Charcot Foot.

25. An apparatus substantially as hereinbefore described having reference to any of the accompanying figures.

26. A method substantially as hereinbefore described having reference to any of the accompanying figures.
